(19) United States
(12) Patent Application Publication
(10) Pub. No.: US 2004/0102444 A1
(43) Pub. Date: May 27, 2004

(54) Method for delivering drugs to the retina

(76) Inventors: Peter Campochiaro, Baltimore, MD (US); Michelle Wong, Duluth, GA (US); Shau-fong Yen, Atlanta, GA (US)

Correspondence Address: THOMAS HOXIE, NOVARTIS, CORPORATE INTELLECTUAL PROPERTY, ONE HEALTH PLAZA 430/2, EAST HANOVER, NJ, 07936-1080, US

(21) Appl. No.: 10/663,464

(22) Filed: Sep. 16, 2003

Related U.S. Application Data
(60) Provisional application No. 60/411,669, filed on Sep. 18, 2002.

(51) Int. Cl. A61K 31/503

(52) U.S. Cl. 514/248

(57) ABSTRACT

The invention relates to methods for the delivery of certain phthalazine derivatives to the retina(s) of a subject in need of treatment.

The invention relates to methods for the delivery of certain phthalazine derivatives to the retina(s) of a subject in need of treatment.

There are several ways to directly administer drugs to the eye, including topical administration to the cornea, periocular injection, and intraocular injection. Topical administration is the least invasive and most easily reversible approach and has been used to treat several diseases of the cornea or anterior segment of the eye. However, topical administration of antibiotics does not effectively treat intraocular infection. Similarly to antibiotics, it has generally been felt in the art that topical administration does not allow attainment of sufficient drug concentrations in the posterior portion of the eye to treat retinal diseases, including choroidal diseases.

The invention is directed to methods for the delivery of a phthalazine derivative to the retina of a subject afflicted with a retinal disease, comprising the topical ocular administration to a subject in need of treatment of an effective amount of an aqueous composition comprising a compound of formula (I) to treat retinal disease afflicting the subject, wherein formula (I) is

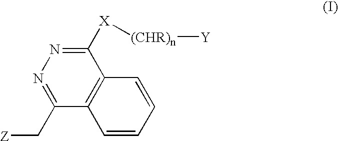

wherein

n is 0 to 2;

R is H or lower alkyl;

X is imino, oxa, or thia;

Y is aryl; and

Z is unsubstituted or substituted pyridyl, an N-oxide thereof, wherein 1 or more N atoms carry an oxygen atom, or a salt thereof.

Subjects suffering from, e.g., retinal or macular edema could benefit from the methods of the present invention, as agents such as those described above can be administered in a topical, non-invasive way to their eyes.

The compounds set out above have been found to be useful in the treatment of ischemic retinopathy, retinal neovascularization, choroidal neovascularization, and retinal and macular edemas.

The general terms used hereinbefore and hereinafter preferably have within the context of this disclosure the following meanings, unless otherwise indicated:

The prefix “lower” denotes a radical having up to and including a maximum of 7, especially up to and including a maximum of 4 carbon atoms, the radicals in question being either linear or branched with single or multiple branching.

Where the plural form is used for compounds, salts and the like, this is taken to mean also a single compound, salt or the like.

Any asymmetric carbon atoms (for example in compounds of formula (I) [or an N-oxide thereof], wherein n=1 and R is lower alkyl) may be present in the (R)—, (S)— or (R,S)—configuration, preferably in the (R)— or (S)—configuration. Substituents at a double bond or a ring may be present in cis- (=Z—) or trans (=E—) form. The compounds may thus be present as mixtures of isomers or as pure isomers, preferably as enantiomer-pure diastereomers.

The index n is preferably 0 or 1, especially 0.

Lower alkyl is especially C₁-C₄-alkyl, e.g., n-butyl, sec-butyl, tert-butyl, n-propyl, isopropyl or especially methyl or also ethyl.

In the preferred embodiment, aryl is an aromatic moiety having 6 to 14 carbon atoms, especially phenyl, naphthyl, fluorenyl or phenanthrenyl, the moieties defined above being unsubstituted or substituted by one or more, preferably up to three, especially one or two substituents, especially selected from amino, mono- or di-substituted amino, halogen, alkyl, substituted alkyl, hydroxy, etherified or esterified hydroxy, nitro, cyano, carboxy, esterified carboxy, alkanoyl, carbamoyl, N-mono- or N,N-disubstituted carbamoyl, amidino, guanidino, mercapto, sulfo, phenylthio, phenyl-lower alkylthio, alkylphenylthio, phenylsulfinyl, phenyl-lower alkylsulfinyl, alkylphenylsulfinyl, phenylsulfonyl, phenyl-lower alkylsulfonyl and alkylphenylsulfonyl, or (as an alternative or in addition to the above group of substituents) selected from lower alkenyl, such as ethenyl, phenyl, lower alkylthio, such as methylthio, lower alkanoyl, such as acetyl, lower alkylmercapto, such as methylmercapto (—S—CH₃), halogen-lower alkylmercapto, such as trifluoromethylmercapto (—S—CF₃), lower alkylsulfonyl, halogen-lower alkylsulfonyl, such as especially trifluoromethane sulfonyl, dihydroxybora (—B(OH)₂), heterocyclyl, and lower alkylene dioxy bound at adjacent C-atoms of the ring, such as methylene dioxy; aryl is preferably phenyl which is either unsubstituted or independently substituted by one or two substituents selected from the group comprising amino; lower alkanoylamino, especially acetylamino; halogen, especially fluorine, chlorine, or bromine; lower alkyl, especially methyl or also ethyl or propyl; halogen-lower alkyl, especially trifluoromethyl; hydroxy; lower alkoxy, especially methoxy or also ethoxy; phenyl-lower alkoxy, especially benzyloxy; and cyano, or (as an alternative or in addition to the previous group of substituents) C₈-C₁₋₂alkoxy, especially n-decyloxy, carbamoyl, lower alkylcarbamoyl, such as n-methyl- or n-tert-butylcarbamoyl, lower alkanoyl, such as acetyl, phenyloxy, halogen-lower alkyloxy, such as trifluoromethoxy or 1,1,2,2-tetrafluoroethyloxy, lower alkoxycarbonyl, such as ethoxycarbonyl, lower alkylmercapto, such as methylmercapto, halogen-lower alkylmercapto, such as trifluoromethylmercapto, hydroxy-lower alkyl, such as hydroxymethyl or 1-hydroxymethyl, lower alkylsulfonyl, such as methane sulfonyl, halogen-lower alkylsulfonyl, such as trifluoromethane sulfonyl, phenylsulfonyl, dihydroxybora (—B(OH)₂), 2-methylpyrimidin-4-yl, oxazol-5-yl, 2-methyl-1,3-dioxolan-2-yl, 1H-pyrazol-3-yl, 1-methyl-pyrazol-3-yl and lower alkylene dioxy bound to two adjacent C-atoms, such as methylene dioxy.

Where mention is made hereinbefore and hereinafter to moieties or substituents as “an alternative or in addition to” the previous group of moieties or substituents, these moieties or substituents and those of the previous group are to be regarded together as one group of substituents from which the respective moieties may be selected, or especially as separate groups. The expression does not mean that one of the radicals following the expression may be added to a member of the previous group by binding. This applies, even if the expression “as an alternative or in addition to” is not mentioned again, for the moieties or substituents, as defined here, in the preferred compounds of formula (I) defined below.

Mono- or di-substituted amino is especially amino substituted by one or two moieties selected independently of one another from lower alkyl, such as methyl; hydroxy-lower alkyl, such as 2-hydroxyethyl; phenyl-lower alkyl; lower alkanoyl, such as acetyl; benzoyl; substituted benzoyl, wherein the phenyl moiety is unsubstituted or especially substituted by one or more, preferably one or two, substituents selected from nitro or amino, or also from halogen, amino, N-lower alkylamino, N,N-di-lower alkylamino, hydroxy, cyano, carboxy, lower alkoxycarbonyl, lower alkanoyl, and carbamoyl; and phenyl-lower alkoxycarbonyl, wherein the phenyl moiety is unsubstituted or especially substituted by one or more, preferably one or two, substituents selected from nitro or amino, or also from halogen, amino, N-lower alkylamino, N,N-di-lower alkylamino, hydroxy, cyano, carboxy, lower alkoxycarbonyl, lower alkanoyl and carbamoyl; and is preferably N-lower alkylamino, such as N-methylamino, hydroxy-lower alkylamino, such as 2-hydroxyethylamino, phenyl-lower alkylamino, such as benzylamino, N,N-di-lower alkylamino, N-phenyl-lower alkyl-N-lower alkylamino, N,N-di-lower alkylphenylamino, lower alkanoylamino, such as acetylamino, or a substituent selected from the group comprising benzoylamino and phenyl-lower alkoxycarbonylamino, wherein the phenyl moiety in each case is unsubstituted or especially substituted by nitro or amino, or also by halogen, amino, N-lower alkylamino, N,N-di-lower alkylamino, hydroxy, cyano, carboxy, lower alkoxycarbonyl, lower alkanoyl or carbamoyl, or as an alternative or in addition to the previous group of moieties by aminocarbonylamino.

Halogen is preferably fluorine, chlorine, bromine or iodine, more preferably fluorine, chlorine or bromine.

In the preferred embodiment, alkyl has up to a maximum of 12 carbon atoms and is preferably lower alkyl, more preferably methyl, ethyl, n-propyl, isopropyl or tert-butyl.

Substituted alkyl is alkyl as last defined, especially lower alkyl, preferably methyl; where one or more, especially up to three, substituents may be present, primarily from the group selected from halogen, especially fluorine, and also from amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

Etherified hydroxy is especially C₈-C₂₀alkyloxy, such as n-decyloxy, lower alkoxy, such as methoxy, ethoxy, isopropyloxy, or n-pentyloxy, phenyl-lower alkoxy, such as benzyloxy or phenyloxy, or as an alternative or in addition to the previous group C₈-C₂₀alkyloxy, such as n-decyloxy, halogen-lower alkoxy, such as trifluoromethyloxy or 1,1,2,2-tetrafluoroethoxy.

Esterified hydroxy is especially lower alkanoyloxy, benzoyloxy, lower alkoxycarbonyloxy, such as tert-butoxycarbonyloxy, or phenyl-lower alkoxycarbonyloxy, such as benzyloxcarbonyloxy.

Esterified carboxy is especially lower alkoxycarbonyl, such as tert-butoxycarbonyl or ethoxycarbonyl, phenyl-lower alkoxycarbonyl or phenyloxycarbonyl.

Alkanoyl is primarily alkylcarbonyl, especially lower alkanoyl, e.g. acetyl.

N-mono- or N,N-disubstituted carbamoyl is especially substituted by one or two substituents, lower alkyl, phenyl-lower alkyl or hydroxy-lower alkyl, at the terminal nitrogen atom.

Alkylphenylthio is especially lower alkylphenylthio.

Alkylphenylsulfinyl is especially lower alkylphenylsulfinyl.

Alkylphenylsulfinyl is especially lower alkylphenylsulfinyl.

Unsubstituted pyridyl is preferably 3- or 4-pyridyl. Specially preferred is 4-pyridyl.

Substituted pyridyl is preferably 3- or 4-pyridyl which is substituted by one or two substituents, in particular selected from lower alkyl, preferably methyl, ethyl; halogen preferably chloro, fluoro, bromo; lower alkyl halides preferably trifluoromethyl; lower alkoxy preferably methoxy, ethoxy; hydroxy; cyano; amino, N-lower alkylamino, N,N-di-lower alkylamino. Specially preferred is 4-pyridyl substituted by methyl, chloro, fluoro, trifluoromethyl or methoxy.

Heterocyclyl is especially a five or six-membered heterocyclic system with 1 or 2 heteroatoms selected from the group comprising nitrogen, oxygen and sulfur, which may be unsaturated or wholly or partly saturated, and is unsubstituted or substituted especially by lower alkyl, such as methyl; a moiety selected from 2-methylpyrimidin-4-yl, oxazol-5-yl, 2-methyl-1,3-dioxolan-2-yl, 1H-pyrazol-3-yl and 1-methyl-pyrazol-3-yl is preferred.

Aryl in the form of phenyl which is substituted by lower alkylene dioxy bound to two adjacent C-atoms, such as methylenedioxy, is preferably 3,4-methylenedioxyphenyl.

An N-oxide of a compound of formula (I) is preferably an N-oxide in which a phthalazine-ring nitrogen or a nitrogen in the pyridin ring carries an oxygen atom, or several of the said nitrogen atoms carry an oxygen atom.

Salts are especially the pharmaceutically acceptable salts of compounds of formula (I) (or an N-oxide thereof).

When a basic group and an acid group are present in the same molecule, a compound of formula (I) (or an N-oxide thereof) may also form internal salts.

In view of the close relationship between the novel compounds in free form and those in the form of their salts, including those salts that can be used as intermediates, for example, in the purification or identification of the novel compounds, any reference to the free compounds hereinbefore and hereinafter is to be understood as referring also to the corresponding salts, as appropriate and expedient.

Definition of Chemical Structures (CGP 79787D (PTK787), CGP 57 148B and CGP 53 716)

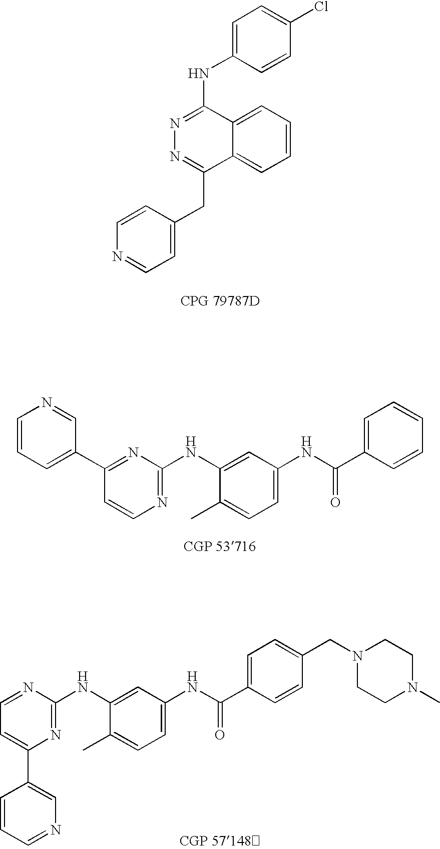

Identification of Relatively Selective Tyrosine Kinase Inhibitors

Table 1 shows the ability of various drugs to inhibit the phosphorylation of poly(GluTyr) by GST-fusion proteins of the kinase domains of several tyrosine kinase receptors using a standard tyrosine kinase assay. PTK 787 is a good inhibitor of phosphorylation by the kinase domains of human VEGF receptors 1 and 2, and PDGF receptor; it is about 10-fold less potent as an inhibitor of mouse VEGF receptor 2 compared to the human receptor, but still inhibits at a reasonable concentration (IC₅₀=290 nM). PTK 787 is a poor inhibitor of phosphorylation by the kinase domain of all other tyrosine kinase and serine/threonine kinases tested (IC₅₀>1 μM, Table 1). CGP 57 148B and CGP 53 716 are potent inhibitors of phosphorylation by the kinase domain of PDGF receptor, BCR ABL and v-ABL, and provide little or no inhibitory activity of other RTKs including VEGF receptors.

TABLE 1
Kinase Inhibitory Profiles (IC₅₀ (μM))

| Tyrosine | | | |
|---|---|---|---|
| Kinase | PTK 787 | CGP 53 716 | CGP 57 148 |
| KDR | 0.13 | >1 | — |
| Flt-1 | 0.34 | >1 | — |
| Flk-1 | 0.49 | >1 | — |
| PDGF-Rβ | 0.2 | 6 | .83 |
| c-Kit | 0.413 | — | — |
| FGF-1 | >50 | — | — |
| Tie-2 | >1 | >1 | — |
| C-Met | >1 | >10 | — |
| EGF-R | 34 | >100 | >100 |
| C-Scr | >10 | >100 | >100 |
| V-Abl | >10 | 0.4 | 0.038 |

In vivo activity requires drugs to enter cells and therefore agents are tested in cellular autophosphorylation assays, using Chinese hamster ovary cells transfected with one of several RTK genes. PTK787 inhibits autophosphorylation of VEGF receptors, PDGF receptors, and c-Kit, but is a poor inhibitor of c-erbB2 or insulin receptor. CGP 57 148B and CGP 53 716 are potent inhibitors of PDGF receptors, but are poor inhibitors of other receptors tested including VEGF receptors. Therefore, PTK 787 inhibits the tyrosine kinase activity of growth factor receptors belonging to the same family (class III), while CGP 57 148B and CGP 53 716 are more selective and only block PDGF receptors.

Preferred Compounds

With the groups of preferred compounds of formula (I) mentioned hereinafter, definitions of substituents from the general definitions mentioned hereinbefore may reasonably be used, for example, to replace more general definitions with more specific definitions or especially with definitions characterized as being preferred.

Preference is given to a compound of formula (I)

wherein

n is 0 or 1;

R is H or lower alkyl, especially H or methyl;

X is imino, oxa or thia;

Y is phenyl, which is unsubstituted or is substituted by one or two substituents independently of one another from the group comprising amino; lower alkanoylamino, especially acetylamino; halogen, especially fluorine, chlorine, or bromine; lower alkyl, especially methyl or also ethyl or propyl; halogen-lower alkyl, especially trifluoromethyl; hydroxy; lower alkoxy, especially methoxy or also ethoxy; phenyl-lower alkoxy, especially benzyloxy; and cyano, or (as an alternative or in addition to the previous group of substituents) lower alkenyl, such as ethenyl, C₈-C₁₂alkoxy, especially n-decyloxy, lower alkoxycarbonyl, such as tert-butoxycarbonyl, carbamoyl, lower alkylcarbamoyl, such as N-methyl- or N-tert-butylcarbamoyl, lower alkanoyl, such as acetyl, phenyloxy, halogen-lower alkyloxy, such as trifluoromethoxy or 1,1,2,2-tetrafluoroethyloxy, lower alkoxycarbonyl, such as ethoxycarbonyl, lower alkylmercapto, such as methylmercapto, halogen-lower alkylmercapto, such as trifluoromethylmercapto, hydroxy-lower alkyl, such as hydroxymethyl, lower alkylsulfonyl, such as methanesulfonyl, halogen-lower alkylsulfonyl, such as trifluoromethanesulfonyl, phenylsulfonyl, dihydroxybora (—B(OH)₂), 2-methylpyrimidin-4-yl, oxazol-5-yl, 2-methyl-1,3-dioxolan-2-yl, 1H-pyrazol-3-yl, 1-methyl-pyrazol-3-yl and lower alkylenedioxy bound to two adjacent C-atoms, such as methylenedioxy, or is also pyridyl, especially 3-pyridyl; especially phenyl, 2-, 3- or 4-aminophenyl, 2-, 3- or 4-acetylaminophenyl, 2-, 3- or 4-fluorophenyl, 2-, 3- or 4-chlorophenyl, 2-, 3- or 4-bromophenyl, 2,3-, 2,4-, 2,5- or 3,4-dichlorophenyl, chlorofluorophenyl, such as 3-chloro-4-fluorophenyl or also 4-chloro-2-fluoroanilino, 2,-3- or 4-methylphenyl, 2-, 3- or 4-ethylphenyl, 2-, 3- or 4-propylphenyl, methylfluorophenyl, such as 3-fluoro-4-methylphenyl, 2-, 3- or 4-trifluoromethylphenyl, 2-, 3- or 4-hydroxyphenyl, 2-, 3- or 4-methoxyphenyl, 2-, 3 or 4-ethoxyphenyl, methoxychlorophenyl, such as 3-chloro-4-methoxycarbonyl, 2-, 3- or 4-benzyloxyphenyl, 2-, 3- or 4-cyanophenyl, or also 2-, 3- or 4-pyridyl; and

Z is 3- or 4-pyridyl, which is unsubstituted or is substituted by one or two substituents independently of one another from the group comprising halogen, especially fluorine, chlorine or bromine; lower alkyl, especially methyl or also ethyl or propyl; halogen-lower alkyl, especially trifluoromethyl; hydroxy; lower alkoxy, especially methoxy or also ethoxy.

Special preference is given to a compound of formula (I),

n is 0 or 1;

R is H;

X is imino;

Y is phenyl, which is unsubstituted or is substituted by one or two substituents independently of one another from the group comprising amino; lower alkanoylamino, especially acetylamino; halogen, especially fluorine, chlorine or bromine; lower alkyl, especially methyl; halogen-lower alkyl, especially trifluoromethyl; hydroxy; lower alkoxy, especially methoxy; phenyl-lower alkoxy, especially benzyloxy; and cyano; especially phenyl, 2-, 3- or 4-aminophenyl, 2-, 3 or 4-acetylaminophenyl, 2-, 3- or 4-fluorophenyl, 2-, 3- or 4-chlorophenyl, 2-, 3- or 4-bromophenyl, 2,3-, 2,4-, 2,5- or 3,4-dichlorophenyl, chlorofluorophenyl, such as 3-chloro-4-fluorophenyl, 2,-3- or 4-methylphenyl, 2-, 3- or 4-trifluoromethylphenyl, 2-, 3- or 4-hydroxyphenyl, 2-, 3- or 4-methoxycarbonyl, methoxychlorophenyl, such as 3-chloro-4-methoxycarbonyl, 2-, 3- or 4-benzyloxyphenyl, or 2-, 3- or 4-cyanophenyl; and Z is 4-pyridyl, which is unsubstituted or is substituted by a substituent from the group consisting of halogen, especially fluorine, chlorine, or bromine; lower alkyl, especially methyl or also ethyl or propyl; halogen-lower alkyl, especially trifluoromethyl; hydroxy; lower alkoxy, especially methoxy.

Special preference is also given to a compound of formula (I),

n is 0 or 1;

R is H;

X is imino;

Y is phenyl, which is unsubstituted or is substituted by one or two substituents independently of one another from the group comprising halogen, especially fluorine, chlorine, or bromine; lower alkyl, especially methyl; halogen-lower alkyl, especially trifluoromethyl; hydroxy; lower alkoxy, especially methoxy; cyano; and

Z is 4-pyridyl, which is unsubstituted or is substituted by a substituent from the group consisting of halogen, especially fluorine, or chlorine; lower alkyl, especially methyl; halogen-lower alkyl, especially trifluoromethyl; hydroxy; lower alkoxy, especially methoxy.

Special preference is also given to a compound of formula (I),

n is 0;

X is imino;

Y is phenyl, which is unsubstituted or is substituted by one substituent selected from the group consisting of fluorine, chlorine; methyl; trifluoromethyl; hydroxy; cyano and methoxy; and

Z is 4-pyridyl, which is unsubstituted or is substituted by a substituent selected from the group consisting of fluorine, or chlorine; methyl; trifluoromethyl; hydroxy; methoxy.

Special preference is also given to a compound of formula (I),

n is 0;

X is imino;

Y is phenyl, which is unsubstituted or is substituted by one substituent selected from the group consisting of fluorine, chlorine; methyl; methoxy; cyano and trifluoromethyl; and

Z is 4-pyridyl, which is unsubstituted or is substituted by a substituent selected from the group consisting of fluorine, or chlorine; and methyl.

High preference is given to a compound selected from the group consisting of:

1-(4-Chloroanilino)-4-(4-pyridylmethyl)phthalazine;

1-(3-Chloroanilino)-4-(4-pyridylmethyl)phthalazine; 1-Anilino-4-(4-pyridylmethyl)phthalazine; 1-Benzylamino-4-(4-pyridylmethyl)phthalazine; 1-(4-Methoxyanilino)-4-(4-pyridylmethyl)phthalazine; 1-(3-Benzyloxyanilino)-4-(4-pyridylmethyl)phthalazine; 1-(3-Methoxyanilino)-4-(4-pyridylmethyl)phthalazine; 1-(2-Methoxyanilino)-4-(4-pyridylmethyl)phthalazine; 1-(4-Trifluoromethylanilino)-4-(4-pyridylmethyl)phthalazine; 1-(4-Fluoroanilino)-4-(4-pyridylmethyl)phthalazine; 1-(3-Hydroxyanilino)-4-(4-pyridylmethyl)phthalazine; 1-(4-Hydroxyanilino)-4-(4-pyridylmethyl)phthalazine; 1-(3-Aminoanilino)-4-(4-pyridylmethyl)phthalazine; 1-(3,4-Dichloroanilino)-4-(4-pyridylmethyl)phthalazine; 1-(4-Bromoanilino)-4-(4-pyridylmethyl)phthalazine; 1-(3-Chloro-4-methoxyanilino)-4-(4-pyridylmethyl)phthalazine; 1-(4-Cyanoanilino)-4-(4-pyridylmethyl)phthalazine; 1-(4-Methylanilino)-4-(4-pyridylmethyl)phthalazine; 1-(3-Chloro-4-fluoroanilino)-4-(4-pyridylmethyl)phthalazine; and 1-(3-Methylanilino)-4-(4-pyridylmethyl)phthalazine.

A compound of the invention, once the structure is know to one of ordinary skill in the art, may be prepared by processes known per se, for example, as described in the working examples infra.

Pharmaceutical Preparations, Methods and Uses

The present invention relates also to aqueous pharmaceutical compositions suitable for topical ocular administration that comprise water and a compound of formula (I) (or an N-oxide thereof) as active ingredient and that can be used especially in the treatment of the diseases mentioned above. The compositions comprise the active ingredient alone or, preferably, together with one or more ophthalmically acceptable carriers and excipients.

Typically, the formulation will comprise polymers, such as hydroxypropylmethyl cellulose, acrylic acid homo- and co-polymers, such as commercially available Carbopols from BF Goodrich, sodium carboxymethylcellulose, carboxymethylcellulose, dextran, polyvinylpyrrolidone or gelatins.

The present invention may further comprise a tonicity enhancing agent. Such tonicity enhancing agents may include compounds, such as urea, glycerol, sorbitol, mannitol, propylene glycol or dextrose. Such tonicity enhancing agent is added to impart an osmolality of approximately 50-500 mOsmol, more preferred from 200-350 mOsmol.

A non-ionic surfactant such as polysorbate 80 (polyoxyethylene(20)sorbitan monooleate) may be incorporated to reduce the cohesion force between drug particles.

Addition of an acceptable buffer system is generally advantageous. Examples of buffer substances include tromethamine (tris-(hydroxymethyl)-aminomethane, TRIS). The pH range is generally in the range of from 4 to 8 and more preferably from 7.0 to 7.8.

The composition may further comprise a preservative, e.g., to inhibit microbial growth upon storage. A non-limiting selection of preservatives includes a quaternary ammonium compound, such as benzalkonium chloride, cetrimide or the like, stabilized oxychloro complexes, such as the commercially available Purite, stabilized perborate, Polyquat or mixtures thereof.

An exemplary pharmaceutical composition of the invention is set forth in Table 2.

TABLE 2

| Ingr dient | % w/v | mg/mL |
|---|---|---|
| PTK 787 | 1.0 | 10 mg/mL |
| Polysorbate 80 | 0.1 | 1.0 mg/mL |
| Carbopol 980 NF | 0.25 | 2.5 mg/mL |
| Hydroxypropylmethyl cellulose | 0.3 | 3.0 mg/mL |
| Sorbitol | 3.43 | 34.3 mg/mL |
| Benzalkonium Chloride NF | 0.015 | 0.15 mg/mL |
| Sodium Hydroxide | Adjust to pH 6.8-7.2 | Adjust to pH 6.8-7.2 |
| Water for Injection | Qs to 100 | Qs to volume |

The following Examples serve to illustrate the invention without limiting the invention in its scope.

Temperatures are measured in degrees celsius. Unless otherwise indicated, the reactions take place at room temperature.

The short forms and abbreviations used have the following definitions:

| | |
|---|---|
| DMSO | dimethyl sulfoxide |
| ESI-MS | electrospray ionization mass spectroscopy |
| Ether | diethyl ether |
| h | hour(s) |
| HV | high vacuum |
| RE | rotary evaporator |
| RT | room temperature |
| m.p. | melting point |
| THF | tetrahydrofuran |

### Example 1

### 1-(4-chloroanilino)-4-(4-pyridylmethyl)phthalazine dihydrochloride

A mixture of 15.22 g (59.52 mmol) 1-chloro-4-(4-pyridylmethyl)phthalazine (for preparation see German Auslegeschrift No. 1 061 788 [published 23 Jul. 1959]), 7.73 g (60.59 mmol) 4-chloroaniline and 200 mL 1-butanol is heated for 2 hours under reflux. The crystallizate which is obtained when the mixture slowly cools to 5° C. is then filtered off and washed with 1-butanol and ether. The filter residue is dissolved in about 200 mL hot methanol, the solution is treated with 0.75 g activated carbon and filtered via a Hyflo Super Cel, and the pH of the filtrate is adjusted to about 2.5 with 7 mL 3N methanolic HCl. The filtrate is evaporated to about half the original volume and ether added until slight turbidity occurs; cooling then leads to the precipitation of crystals. The crystallizate is filtered off, washed with a mixture of methanol/ether (1:2) as well as ether, dried for 8 hours at 110° C. under HV, and equilibrated for 72 hours at 20° C. and in room atmosphere. In this way, the title compound is obtained with a water content of 8.6%; m.p. >270° C.; ¹H NMR (DMSO-d₆) 11.05-12.20 (br), 9.18-9.23 (m, 1H), 8.88 (d, 2H), 8.35-8.40 (m, 1H), 8.18-8.29 (m, 2H), 8.02 (d, 2H), 7.73 (d, 2H), 7.61 (d, 2H), 5.02 (s, 2H); ESI-MS: (M+H)⁺=347.

### Example 2

### 1-(4-chloroanilino)-4-(4-pyridylmethyl)phthalazine hydrochloride

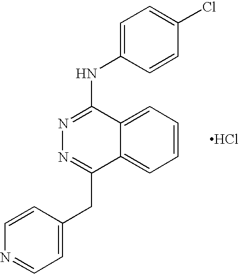

A mixture of 0.972 g (3.8 mmol) 1-chloro-4-(4-pyridylmethyl)phthalazine, 0.656 g (4 mmol) 4-chloroaniline hydrochloride (Research Organics, Inc., Cleveland, Ohio, USA) and 20 mL ethanol is heated for 2 hours under reflux. The reaction mixture is cooled in an ice bath, filtered and the crystallizate washed with a little ethanol and ether. After drying under HV for 8 hours at 110° C. and for 10 hours at 150° C., the title compound is obtained as a result of thermal removal of HCl; m.p. >270° C.; ¹H NMR (DMSO-d₆) 9.80-11.40 (br), 8.89-8.94 (m, 1H), 8.67 (d, 2H), 8.25-8.30 (m, 1H), 8.06-8.17 (m, 2H), 7.87 (d, 2H), 7.69 (d, 2H), 7.49 (d, 2H), 4.81 (s, 2H); ESI-MS: (M+H)⁺=347.

### Example 3

### 1-(4-chloroanilino)-4-(4-pyridylmethyl)phthalazine hydrochloride

A mixture of 1.28 g (5 mmol) 1-chloro-4-(4-pyridylmethyl)phthalazine, 0.67 g (5.25 mmol) 4-chloroaniline and 15 mL 1-butanol is heated for 0.5 hour at 100 hours while stirring in a nitrogen atmosphere. The mixture is then cooled to RT, filtered and the filtrate washed with 1-butanol and ether. For purification, the crystallizate is dissolved in 40 mL of hot methanol, the solution treated with activated carbon, filtered via Hyflo Super Cel and the filtrate evaporated to about half its original volume, resulting in the formation of a crystalline precipitate. After cooling to 0° C., filtration, washing of the filter residue with ether, and drying under HV for 8 hours at 130° C., the title compound is obtained; m.p. >270° C.; ¹H NMR (DMSO-d₆) 9.80-11.40 (br), 8.89-8.94 (m, 1H), 8.67 (d, 2H), 8.25-8.30 (m, 1H), 8.06-8.17 (m, 2H), 7.87 (d, 2H), 7.69 (d, 2H), 7.49 (d, 2H), 4.81 (s, 2H); ESI-MS: (M+H)⁺=347.

### Example 4

### 1-(4-chloroanilino)-4-(4-pyridylmethyl)phthalazine

A mixture of 14.19 g (0.1 mol) phosphorus pentoxide, 13.77 g (0.1 mol) triethylamine hydrochloride and 12.76 g (0.1 mol) 4-chloroaniline is heated and stirred in a nitrogen atmosphere at 200° C. until a homogeneous melt has formed (about 20 minutes). To the melt, 5.93 g (0.025 mol) 4-(4-pyridylmethyl)-1 (2H)-phthalazinone (for preparation see German Auslegeschrift No. 1 061 788 [published 23.07.1959]) is added, and the reaction mixture is stirred for 3 hours at 200° C. After the reaction mixture has cooled to about 100° C., 200 mL of water is added. Stirring is continued until the temperature reaches about 30° C., and then 20 mL conc. ammonia (30% aqueous ammonium hydroxide solution) and 900 mL chloroform are added consecutively. As soon as a diphasic mixture has formed, the organic phase is separated off, dried over anhydrous sodium sulfate, filtered and the filtrate evaporated on a RE to a volume of about 50 mL, to which 100 mL acetate is then added, and the mixture is cooled in an ice bath. The crystallizate obtained is filtered off and washed with acetate and ether. After re-crystallization from methanol and drying under HV for 8 hours at 120° C., the title compound is obtained; m.p. 194-195° C.; ESI-MS: (M+H)⁺=347.

### Example 5

### Delivery of PTK787 to the Retina by Topical Administration

Adult C57BL/6 mice are given topical administration of PTK787 or vehicle twice a day for 3 days and then they are given an intravitreous injection of VEGF in each eye. Intraocular injections are performed with a Harvard pump microinjection apparatus and pulled glass micropipets. Each micropipet is calibrated to deliver 1 μL of vehicle upon depression of a foot switch. Mice are anesthetized with 25 mg/kg of ketamine and 4 mg/kg of xylazine, pupils are dilated with 1% tropicamide. Under a dissecting microscope, the sharpened tip of a micropipet is passed through the sclera just behind the limbus into the vitreous cavity, and the foot switch is depressed and 1 μL of 10⁻⁶ M human VEGF (R&D Systems, Minneapolis, Minn.) is injected.

Measurement of BRB Breakdown Using [³H]Mannitol as Tracer

Six or 24 hours after intraocular injections, mice are given an intraperitoneal injection of 1 μCi/g body weight of [³H]mannitol (New England Nuclear, Boston, Mass.). After one hour, mice are sacrificed and eyes are removed. The cornea and lens are removed and the entire retina is carefully dissected from the eyecup and placed within pre-weighed scintillation vials. The thoracic cavity is opened and the left superior lobe of the lung is removed and placed in another pre-weighed scintiliation viai. A left dorsai incision is made and the retroperitoneal space is entered without entering the peritoneal cavity. The renal vessels are clamped with a forceps and the left kidney is removed, cleaned of all fat, and placed into a pre-weighed scintillation vial. All liquid is removed from the vials and remaining droplets are allowed to evaporate over 20 minutes. The vials are weighed and the tissue weights are recorded. One mL of NCSII solubilizing solution (Amersham, Chicago, Ill.) is added to each vial and the vials are incubated overnight in a 50° C. water bath. The solubilized tissue is brought to RT and decolorized with 20% benzoyl peroxide in toluene in a 50° C. water bath. The vials are brought to RT and 5 mL of Cytoscint ES (ICN, Aurora, OH) and 30 μL of glacial acetic acid are added. The vials are stored for several hours in darkness at 4° C. to eliminate chemoluminescence. Radioactivity is counted with a Wallac 1409 Liquid Scintillation Counter (Gaithersburg, Md.).

Intraocular injection of VEGF result in significant breakdown of the BRB assessed by leakage of [³H]mannitol into the eye. Mice treated with PTK787 suspension in one eye twice a day for 3 days prior to intraocular injection of VEGF show a significantly lower retina to lung leakage ratio (RLLR) compared to eyes of animals that are not treated. There was not a significant difference in RRLR compared to contralateral eyes in the same mice.

What is claimed is:

1. A method for the delivery of a phthalazine derivative to the retina of a subject afflicted with a retinal disease, comprising the topical ocular administration to a subject in need of treatment of an effective amount of an aqueous composition comprising a compound of formula (I) to treat retinal disease afflicting the subject, wherein formula (I) is 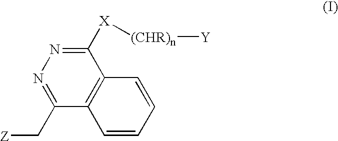
wherein
n is 0 to 2,
R is H or lower alkyl;
X is imino, oxa, or thia;
Y is aryl; and
Z is unsubstituted or substituted pyridyl, an N-oxide thereof, wherein 1 or more N atoms carry an oxygen atom, or a salt thereof.

2. The method of claim 1, wherein said retinal disease is selected from the group consisting of macular edema, choroidal neovascularization and retinal neovascularization.

3. The method of claim 1, wherein said retinal disease is exudative age related macular degeneration.

4. The method of claim 1, wherein said retinal disease is proliferative diabetic retinopathy.

5. The method of claim 1, wherein said retinal disease is an ischemic retinopathy.

6. The method of claim 1 wherein said subject is a human.

7. The method of claim 1,
wherein
n is 0 or 1;
R is H or lower alkyl;
X is imino, oxa or thia;
Y is phenyl, lower alkenyl, lower alkoxycarbonyl, lower alkylcarbamoyl, lower alkanoyl, phenyloxy, halogen-lower alkyloxy, lower alkoxycarbonyl, lower alkylmercapto, halogen-lower alkylmercapto, hydroxy-lower alkyl, lower alkylsulfonyl, phenylsulfonyl, halogen-lower alkylsulfonyl, dihydroxybora (—B(OH)₂), 2-methylpyrimidin-4-yl, oxazol-5-yl, 2-methyl-1,3-dioxolan-2-yl, 1H-pyrazol-3-yl, 1-methyl-pyrazol-3-yl, lower alkylenedioxy bound to two adjacent C-atoms, pyridyl, or 4-chloro-2-fluoroanilino, wherein said phenyl is unsubstituted or is substituted by one or two substituents independently of one another from the group comprising amino, lower alkanoylamino, halogen, lower alkyl, halogen-lower alkyl, hydroxy, lower alkoxy, phenyl-lower alkoxy and cyano; and
Z is 3- or 4-pyridyl, lower alkyl, halogen-lower alkyl, lower alkoxy or hydroxy, wherein said pyridyl is unsubstituted or is substituted by one or two substituents independently of one another from the group comprising halogen.

8. The method of claim 1,
wherein
n is 0 or 1;
R is H;
X is imino;
Y is phenyl, lower alkanoylamino, halogen, lower alkyl, halogen-lower alkyl, hydroxy, lower alkoxy, phenyl-lower alkoxy or cyano, wherein said phenyl is unsubstituted or is substituted by one or two substituents independently of one another from the group comprising amino; and
Z is 4-pyridyl, lower alkyl, halogen-lower alkyl, hydroxy and lower alkoxy, wherein pyridyl is unsubstituted or is substituted by a substituent from the group consisting of halogen.

9. The method of claim 1,
wherein
n is 0 or 1;
R is H;
X is imino;
Y is phenyl, lower alkyl, halogen-lower alkyl, hydroxy; lower alkoxy or cyano, wherein phenyl is unsubstituted or is substituted by one or two substituents independently of one another from halogen; and
Z is 4-pyridyl, lower alkyl, halogen-lower alkyl, hydroxy or lower alkoxy, wherein said pyridyl is substituted or unsubstituted by a halogen.

10. The method of claim 1,
wherein
n is 0;
X is imino;
Y is phenyl, methyl, trifluoromethyl, hydroxy, cyano or methoxy, wherein said phenyl is substituted or unsubstituted by fluorine or chlorine; and
Z is 4-pyridyl, methyl, trifluoromethyl, hydroxy or methoxy, wherein said pyridyl is substituted or unsubstituted by fluorine or chlorine.

11. The method of claim 1,
wherein
n is 0;
X is imino;
Y is phenyl, methyl, methoxy, cyano or trifluoromethyl, wherein said phenyl is substituted or unsubstituted by chlorine or fluorine; and
Z is 4-pyridyl or methyl, wherein said pyridyl is substituted or unsubstituted by chlorine or fluorine.

12. The method of claim 1, wherein said compound is selected from the group consisting of:
1-(4-Methylanilino)-4-(4-pyridylmethyl)phthalazine;
1-(3-Chloroanilino)-4-(4-pyridylmethyl)phthalazine;
1-Anilino-4-(4-pyridylmethyl)phthalazine;
1-Benzylamino-4-(4-pyridylmethyl)phthalazine;
1-(4-Methoxyanilino)-4-(4-pyridylmethyl)phthalazine;
1-(3-Benzyloxyanilino)-4-(4-pyridylmethyl)phthalazine;
1-(3-Methoxyanilino)-4-(4-pyridylmethyl)phthalazine;
1-(2-Methoxyanilino)-4-(4-pyridylmethyl)phthalazine;
1-(4-Trifluoromethylanilino)-4-(4-pyridylmethyl)phthalazine;
1-(4-Fluoroanilino)-4-(4-pyridylmethyl)phthalazine;
1-(3-Hydroxyanilino)-4-(4-pyridylmethyl)phthalazine;
1-(4-Hydroxyanilino)-4-(4-pyridylmethyl)phthalazine;
1-(3-Aminoanilino)-4-(4-pyridylmethyl)phthalazine;
1-(3,4-Dichloroanilino)-4-(4-pyridylmethyl)phthalazine;
1-(4-Bromoanilino)-4-(4-pyridylmethyl)phthalazine;
1-(3-Chloro-4-methoxyanilino)-4-(4-pyridylmethyl)phthalazine;
1-(4-Cyanoanilino)-4-(4-pyridylmethyl)phthalazine;
1-(3-Chloro-4-fluoroanilino)-4-(4-pyridylmethyl)phthalazine;
1-(3-Methylanilino)-4-(4-pyridylmethyl)phthalazine; and
pharmaceutically acceptable salts thereof.

13. The method of claim 1, wherein said compound is 1-(3-Chloroanilino)-4-(4-pyridylmethyl)phthalazine and said subject is a human.

14. The method of claim 13, wherein said retinal disease is selected from the group consisting of macular edema, choroidal neovascularization and retinal neovascularization.

15. The method of claim 13, wherein said retinal disease is exudative age related macular degeneration.

16. The method of claim 14, wherein said retinal disease is proliferative diabetic retinopathy.

17. The method of claim 14, wherein said retinal disease is an ischemic retinopathy.

18. A composition comprising a squeezable container suitable for dispensing drops of an aqueous solution, and further comprising disposed within said container an aqueous composition comprising water and a compound of formula (I) to treat retinal disease afflicting the subject, wherein formula (I) is 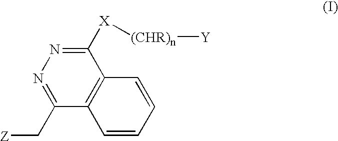
wherein
n is 0 to 2;
R is H or lower alkyl;
X is imino, oxa, or thia;
Y is aryl; and
Z is unsubstituted or substituted pyridyl, an N-oxide thereof, wherein 1 or more N atoms carry an oxygen atom, or a salt thereof.

19. The composition of claim 18, wherein said compound is selected from the group consisting of:
1-(4-Methylanilino)-4-(4-pyridylmethyl)phthalazine;
1-(3-Chloroanilino)-4-(4-pyridylmethyl)phthalazine;
1-Anilino-4-(4-pyridylmethyl)phthalazine;
1-Benzylamino-4-(4-pyridylmethyl)phthalazine;
1-(4-Methoxyanilino)-4-(4-pyridylmethyl)phthalazine;
1-(3-Benzyloxyanilino)-4-(4-pyridylmethyl)phthalazine;
1-(3-Methoxyanilino)-4-(4-pyridylmethyl)phthalazine;
1-(2-Methoxyanilino)-4-(4-pyridylmethyl)phthalazine;
1-(4-Trifluoromethylanilino)-4-(4-pyridylmethyl)phthalazine;
1-(4-Fluoroanilino)-4-(4-pyridylmethyl)phthalazine;
1-(3-Hydroxyanilino)-4-(4-pyridylmethyl)phthalazine;
1-(4-Hydroxyanilino)-4-(4-pyridylmethyl)phthalazine;
1-(3-Aminoanilino)-4-(4-pyridylmethyl)phthalazine;
1-(3,4-Dichloroanilino)-4-(4-pyridylmethyl)phthalazine;
1-(4-Bromoanilino)-4-(4-pyridylmethyl)phthalazine;
1-(3-Chloro-4-methoxyanilino)-4-(4-pyridylmethyl)phthalazine;
1-(4-Cyanoanilino)-4-(4-pyridylmethyl)phthalazine;
1-(3-Chloro-4-fluoroanilino)-4-(4-pyridylmethyl)phthalazine;
1-(3-Methylanilino)-4-(4-pyridylmethyl)phthalazine; and pharmaceutically acceptable salts thereof.
